(19) United States
(12) Patent Application Publication
(10) Pub. No.: US 2018/0243448 A1
(43) Pub. Date: Aug. 30, 2018

(54) DEVICES, KITS, AND METHODS FOR DETERMINING INEFFECTIVENESS OF ANESTHETICS

(71) Applicant: AlkaliDx, Inc., Chestnut Hill, MA (US)

(72) Inventor: Michael M. SEGAL, Chestnut Hill, MA (US)

(21) Appl. No.: 15/755,127

(22) PCT Filed: Aug. 26, 2016
(86) PCT No.: PCT/US16/48990

§ 371 Date: Feb. 26, 2018

Related U.S. Application Data
(60) Provisional application No. 62/210,747, filed on Aug. 27, 2015.

(51) Int. Cl. A61K 49/00; A61K 9/00; A61K 31/167; A61K 31/245; A61K 31/585; A61K 31/522; A61B 5/16; A61B 5/00; A61B 5/01

(52) U.S. Cl. CPC A61K 49/0006; A61K 9/006; A61K 31/167; A61K 31/245; A61K 31/585; A61B 5/0053; A61B 5/168; A61B 5/4848; A61B 5/4017; A61B 5/483; A61B 5/01; A61K 31/522

(57) ABSTRACT

In general, the invention provides kits, devices, and methods for determining the ineffectiveness of an anesthetic, (e.g., lidocaine), using a topical approach that avoids injection. The methods typically employ the placement of aliquots of two different formulations, at least one including an anesthetic, in different locations on a subject. Further embodiments may employ a single formulation including the anesthetic.

### BACKGROUND OF THE INVENTION

The invention relates to the fields of devices and kits for determining the ineffectiveness of anesthetics in individuals and to methods of treatment of attentional disorders and premenstrual syndrome.

There is debate in the medical community regarding the nature of attentional disorders, such as ADHD (Attention Deficit Hyperactivity Disorder) or ADD (Attention Deficit Disorder). In particular, many diseases and states may lead to the finding of attention deficit. While current behavioral testing can identify individuals with the finding of attention deficit, this testing does not identify the underlying cause or nature of the disease or state.

Certain anesthetics may also be ineffective in certain individuals, potentially resulting in unnecessary pain or discomfort during a medical, dental, or surgical procedure. Furthermore, ineffectiveness of certain anesthetics in an individual may be a biomarker for certain forms of attentional disorder.

Accordingly, there is a need for new methods to assess the effectiveness of anesthetics and also to identify forms of attention deficit.

### SUMMARY OF THE INVENTION

In general, the invention provides kits, devices, and methods for determining the ineffectiveness of an anesthetic, (e.g., lidocaine), using a topical approach that avoids injection. The methods typically employ the placement of aliquots of two different formulations, at least one including an anesthetic, in different locations on a subject. Further methods may employ a single formulation including the anesthetic. The methods may be practiced with any of the kits and devices described herein. The invention allows for several components:
1. Determination of effectiveness: by use of a second formulation, which can be a “reference” anesthetic that is assumed to be effective, (e.g., benzocaine, articaine, bupivacaine, or mepivacaine) or a “control” such as the base un-medicated gel for lidocaine that is assumed to be ineffective;
2. Visual indication of the formulations so that adequate coverage can be determined reliably;
3. Applicators and application technique include either an integrated applicator that separates the two formulations or two independent applicators and various procedures to ensure effective application;
4. Metrics of effectiveness, include tactile sensations, such as numbness, temperature sensitivity, or taste; and
5. Double-blind mechanism for determining the effectiveness, thus increasing the confidence in a test requiring patient reported outcomes.

The kit may be used to ascertain whether a particular anesthetic will work for a given patient. It can also be used to diagnose an attentional disorder, e.g., a channelopathy form of ADHD or ADD, premenstrual syndrome (PMS), as well as some related conditions.

Determination of Effectiveness

Reference Model:

In the reference model, the invention provides a kit with an aliquot of a first formulation including a first anesthetic (e.g., lidocaine) and formulated for topical, (e.g., in the mouth, known as buccal) administration; an aliquot of a second formulation including a second anesthetic (e.g., benzocaine, articaine, bupivacaine, or mepivacaine) and formulated for topical, (e.g., buccal), administration. Other first or second anesthetics include butamben, dibucaine, oxybuprocaine, pramoxine, procaine, proparacaine, proxymetacaine, and tetracaine.

Control Model:

In the control model, the kit provides an aliquot of a first formulation including the first anesthetic (e.g., lidocaine) and formulated for topical, e.g., buccal, administration and an aliquot of a second formulation not including an anesthetic, (e.g., a control base without the medication), and formulated for topical, (e.g., buccal), administration.

Visual Indication of the Formulations

The aliquot of the first formulation and the aliquot of the second formulation will be visually distinguishable, allowing the user to check that the aliquots have properly covered the target area. Suitable visual indicators include color, reflectivity, light scattering, opacity, or inclusion of particles (e.g., colored or reflective beads or flakes). Each aliquot will have some visual distinction so that the tester can check that each has been applied properly covered the target area.

Applicator and Application Technique

In certain embodiments, the first and second locations are in the mouth of the subject, (e.g., from the lip, (e.g., upper lip), to the gum, (e.g., upper gum)). In other embodiments, the locations are on both sides of the tongue, (i.e., left and right). A physical barrier configured to adhere topically to a subject may be employed to cover the location of administration of one or both of the first and second locations. In certain embodiments, the physical barrier includes an absorbent material, (e.g., gauze).

Integrated Applicator Model:

In the integrated applicator model, the invention provides a device including a body having first and second regions, where the first and second regions are physically separated; an aliquot of the first formulation (e.g., including lidocaine) and the second formulation (e.g., not including an anesthetic). In certain embodiments, the body includes a strip having a primary face, and the first and second regions are disposed on the primary face. Such a strip may also include at least one barrier located between the first and second regions. In certain embodiments, the body is configured for placement in the mouth of a subject; for example, the body is configured for placement from the lip, (e.g., upper lip), to the gum, (e.g., upper gum). When configured for use in the mouth, the body may be shaped to accommodate a buccal frenulum. In other embodiments, the body is configured placement of the first and second regions on the sides of the tongue (i.e., left and right). In certain embodiments, the body further includes a protective sheet covering the first and/or second region, wherein the protective sheet is removed prior to use.

Independent Applicators Model:

In the independent applicators model, the first formulation is provided separately from the second formulation, (e.g., each formulation on a gauze pad or swab).

Metrics of Effectiveness

Determining the effectiveness occurs after administration of the first and second formulations, (e.g., up to 2 minutes after).

Tactile Sensation:

Tactile sensation can be determined at the first and second locations. When the second formulation includes the second anesthetic, a difference in the tactile sensation between the locations indicates that the first anesthetic is ineffective in the subject. When the second formulation does not include an anesthetic, the lack of a difference between the test and control regions indicates that the first anesthetic is ineffective in the subject. In certain embodiments, the determining may include questioning the subject regarding feeling of numbness, puffiness, or pins and needles. Alternatively, the determining includes applying pressure or a pin prick to the first and second regions.

Temperature Sensitivity:

When using temperature sensitivity, the method includes contacting the locations with a probe having a temperature different from the body temperature, (e.g., at least 10 degrees warmer or colder). Kits may include two probes, one for each location. For example, the method may include refrigeration of a probe made from high thermal conductivity (e.g., metal) to ˜32 degrees Fahrenheit and application of the probe to the locations in the mouth treated with two formulations. In certain embodiments, the determining may include questioning the subject about which side is colder or warmer.

Taste Sensitivity:

When using taste sensitivity, the method includes contacting locations on the tongue with a taste agent, e.g. a liquid, gel, or soluble tablet, that provides a distinctive taste, e.g., sweet, sour, salty, or bitter. Kits of the invention may thus include such taste agents. The determining may include questioning the subject about differences in taste in the locations.

For each of tactile, temperature, or taste, the invention may include probes. Probes may individual or integrated to reach locations simultaneously, as described herein for the formulations.

Double-Blind Mechanism for Determining Effectiveness

Directions on how the test subject is to indicate the side where they feel the effect (see FIG. 4), together with a recording mechanism that avoids confusion about left and right (for example, allowing the kit to be used with younger children) and my left/your left confusion between subject and user may be included in the invention. Furthermore, a peel off or scratch off “key” that indicates the interpretation for each of the 4 possible responses for this particular combination of materials and left-right orientations of the kit may be provided, allowing the user to collect the responses in a manner that is double-blinded.

### Single Formulation Embodiments

In addition to the above embodiments that employ two formulations, the invention may also be employed with a single formulation including the first anesthetic. In these embodiments, the formulation may include a visual indicator, as described herein. The formulation may be applied by any suitable device as described herein, e.g., swab or gauze pad. Determination of effectiveness may be performed using any of the metrics provided herein, tactile, temperature, or taste sensitivity. In these embodiments, the contacting of the location for tactile, temperature, or taste sensitivity can be performed only on the location treated with the formulation or on both the location of the formulation and an untreated location, as described herein when two formulations are employed.

### Uses for the Invention

The invention has several applications.

Anesthesia:

The invention provides for methods of determining the appropriate anesthetic for use in various procedures. If the subject is insensitive to the first anesthetic, (e.g., lidocaine), the method includes administering another anesthetic to the subject prior to a medical, dental, or surgical procedure.

Diagnosing an Attention Disorder-Related Channelopathy.

The invention provides for methods of determining types of attentional disorder, e.g., where the subject is diagnosed with an attentional disorder, (e.g., ADD or ADHD). If the subject diagnosed with an attentional disorder is insensitive to the first anesthetic, (e.g., lidocaine), the invention may further include treatment for the channelopathy form of attention disorders, hypokalemic sensory overstimulation, including administering a low sugar and/or low sodium diet to the subject, and/or administering potassium or drugs that modulate levels of potassium to the subject.

The term “attentional disorder” refers to a condition characterized by inattention, over-activity, and/or impulsiveness. Attentional disorders include, without limitation, Attention Deficit Hyperactivity Disorder, Attention Deficit Disorder, and Hyperkinetic Disorder. Attention Deficit Hyperactivity Disorder, which is also referred to in the literature as Attention Deficit Disorder/Hyperactivity Syndrome (ADD/HS), is a condition (or group of conditions) characterized by impulsiveness, distractibility, inappropriate behavior in social situations and hyperactivity. Other disorders may include a finding of attention deficit, such as Asperger syndrome, and are included in this definition.

Premenstrual Syndrome:

The invention provides for method of treatment of premenstrual syndrome (PMS). If the subject is diagnosed with premenstrual syndrome and is insensitive to the first anesthetic, (e.g., lidocaine), the invention may further include treatment for the channelopathy form of PMS, including administering potassium or drugs that modulate levels of potassium to the subject.

The term “premenstrual syndrome” refers to a combination of physical and emotional disturbances that occur after a woman ovulates and ends with menstruation.

The term “treating” refers to obtaining beneficial or desired results, such as clinical results. Beneficial or desired results can include, but are not limited to, alleviation of one or more symptoms or conditions; diminishment of extent of disease, disorder, or condition; stabilized (i.e., not worsening) state of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

### BRIEF DESCRIPTION OF THE DRAWINGS

FIGS. 1A-1D are illustrations of strips (100) including two formulations (101, 102) (A); a cutout for the buccal frenulum (103) (B); and a barrier between the two formulations (104) (C and, in profile, D).

FIGS. 2A-2C are illustrations of devices with handles in a Y-shape (200) including two formulations (201, 202) (A); T-shape (203) (B); and with two formulations (201, 202) on the tips of a Y-shape (200) (C).

FIG. 3 is an illustration of two independent applicators. A single application can be used in embodiments where only one formulation is employed.

FIG. 4 is an illustration of the collection of results mechanism that avoids left/right confusion by the test subject and the tester.

### DETAILED DESCRIPTION

The present invention provides devices, kits, and methods of determining the ineffectiveness of an anesthetic (e.g., lidocaine) in subjects. Identifying such individuals is often difficult because simple, topical administration of an anesthetic, (e.g., lidocaine), to a subject may not allow for accurate determination of the effectiveness of the anesthetic. Applying the anesthetic directly without a double-blind technique creates the risk of the user influencing the outcome, as the determination requires patient-reported outcomes.

A second complication is that individuals, especially children, may not have experience with anesthesia and be unable to describe when an area is numb; this lack of familiarity with numbness is complicated when the administration is performed topically as the areas underlying and surrounding the site of administration are still capable of feeling. Numbness, (e.g., from lidocaine), is also time delayed in some individuals, and lack of numbness immediately after administration may not be indicative of true effectiveness. Finally, numbness from anesthetics, (e.g., lidocaine), may also present differently in individuals, (e.g., pins and needles or puffiness versus no sensation), making a true determination of effectiveness difficult.

The present invention includes one approach that solves these problems by testing temperature sensitivity in a double-blind way against the two sides of the tongue, the area found to most reliable. Thus, some devices, kits, and methods employ a refrigerated kit with one aliquot of a first formulation including an anesthetic and a second formulation not including an anesthetic for comparison of feeling of cold when two cold thermally conductive pieces are applied, where NO difference in the sensation of cold would indicate the ineffectiveness of the anesthetic.

These devices and kits can generally be used to determine the effectiveness of a particular anesthetic prior to a medical, surgical, or dental procedure. In addition, the lack of effectiveness of certain anesthetics, (e.g., lidocaine), is a diagnostic criterion for certain forms of attentional disorder and premenstrual syndrome.

Devices and Kits

The devices and kits of the invention may include several components:
1. Determination of effectiveness by use of a second formulation, which can be a “reference” anesthetic that is assumed to be effective, (e.g., benzocaine, articaine, bupivacaine, or mepivacaine) or a “control” such as the base un-medicated gel for lidocaine that is assumed to be ineffective;
2. Visual indication of the formulations so that adequate coverage can be determined reliably;
3. Applicators and application technique can include either an integrated applicator that separates the two formulations or two independent applicators and various procedures to ensure effective application;
4. Metrics of effectiveness, include tactile sensations, such as numbness, temperature sensitivity, or taste sensitivity; or
5. Double-blind mechanism for determining the effectiveness, thus increasing the confidence in a test requiring patient reported outcomes.

The kit or device may be used to ascertain whether a particular anesthetic will work for a given patient. It can also be used to diagnose attentional disorders, e.g., a channelopathy form of ADHD (Attention Deficit Hyperactivity Disorder) or ADD (Attention Deficit Disorder), premenstrual syndrome (PMS), as well as some related conditions.

Determination of Effectiveness.

In the “reference model” embodiment, it is typically known that one of the anesthetics is effective in the subject, which is used as “reference” for a positive response to compare to the other anesthetic being tested for effectiveness. Suitable first anesthetics include lidocaine, benzocaine, articaine, bupivacaine, butamben, dibucaine, mepivacaine, oxybuprocaine, pramoxine, procaine, proparacaine, proxymetacaine, or tetracaine, and suitable second anesthetics include benzocaine, articaine, bupivacaine, butamben, dibucaine, mepivacaine, oxybuprocaine, pramoxine, procaine, proparacaine, proxymetacaine, or tetracaine. Preferably, the anesthetic being tested for effectiveness is lidocaine. Preferably the anesthetics will be formulated for oral administration. The reference anesthetic is preferably benzocaine, articaine, bupivacaine, or mepivacaine. In the reference model, a difference in tactile, temperature, or taste sensitivity indicates that the first anesthetic is ineffective for the subject.

In the “control model” embodiment, the second formulation does not include an anesthetic, e.g., a control base without the medication. The control model has the advantage of not needing to know if other anesthetics are normally effective for the patient, when testing the first anesthetic, while at the same time preserving the double-blind nature of the test, e.g., by having both formulations have the same “feel” in the mouth. Here too, preferably the anesthetics will be formulated for oral administration. In the control model, no difference in tactile, temperature, or taste sensitivity indicates that the first anesthetic is ineffective for the subject.

Visual Indication of the Formulations:

The user may need a mechanism to ensure adequate coverage of both formulations, which is achieved by providing a visual indicator in each formulation. Suitable visual indicators include color, reflectivity, light scattering, opacity, or inclusion of particles (e.g., colored or reflective beads or flakes). To mitigate the risk that the indicator, e.g., color, is associated in the mind of the tester with an outcome and that the tester influences the subject's response, the indicator, e.g., color, of first anesthetic, (e.g., lidocaine), may alternate between indicator, e.g., color, 1 and indicator, e.g., color, 2. A box of kits, as ordered by a medical professional may contain a random mix of kits where the first anesthetic has indicator, e.g., color, 1 and where the first anesthetic has indicator, e.g., color 2.

Applicators and Application Technique:

Devices of the invention will preferably be inserted into the mouth of the subject as this generates the most reliable results, and are preferably configured for use on each side of the tongue for the same reason. Devices may, however, be configured for use on the area from the lip to the gum or cheek. Devices of the invention include “integrated” applicators containing both formulations, and “independent” applicators each containing one of the formulations. In the embodiment of the integrated applicator, the aliquots of the first formulation and second formulations, e.g., either containing a second anesthetic or control un-medicated gel for the first anesthetic, are placed on the body of a device. The formulations are spaced apart on the body of the device to prevent mixing when applied to the subject and to allow sufficient spatial separation for the subject to discern a difference in tactile, temperature, or taste sensitivity. The spacing between the two formulations is typically either side of the tongue, although in some embodiments it could be under the upper lip on either side of buccal fold. Bodies may be formed of any suitable material, such as wood, metal, heavy paper or plastic. Devices may also include a barrier that may or may not be absorbent between the two formulations. Such barriers may aid in preventing the formulations from mixing during administration.

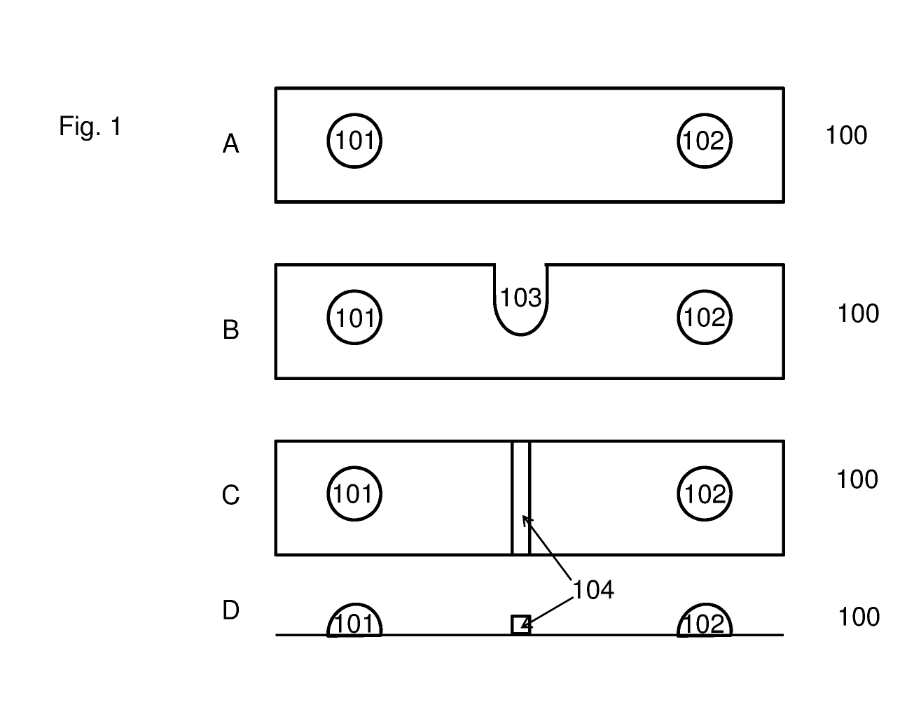

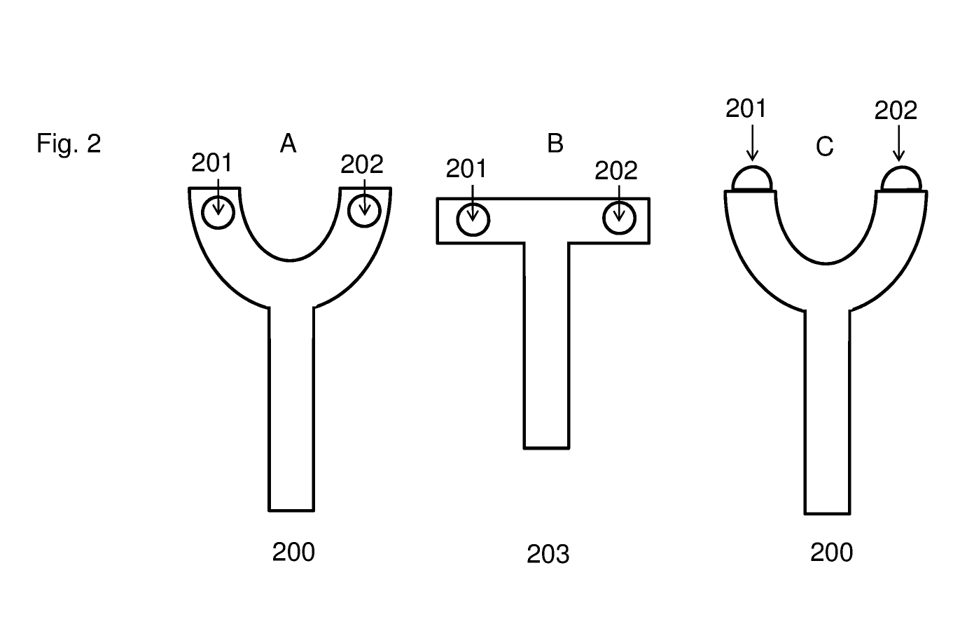

In another embodiment, the integrated device is a flexible strip where the two formulations are placed on the same face of the strip (FIG. 1A-1D). The device may be a thin strip, sized to fit comfortably between the gum and lip, (e.g., upper lip), where the two formulations are spaced apart on the strip. The strip may include a cutout to accommodate the buccal frenulum (FIG. 1B) and/or a physical barrier to reduce possible mixing of the two formulations (FIGS. 1C-1D). In another embodiment, the body of the device includes a handle and an application region, (e.g., with a Y- or T-shape (FIGS. 2A-2B)). Typically, the formulations are placed on the same face of the body, but can be placed on opposite faces or on both faces. Formulations may also be placed on the tip ends of a Y- or U-shaped body (FIG. 2C). The integrated applicator may also be covered with a protective film that can be removed prior to use.

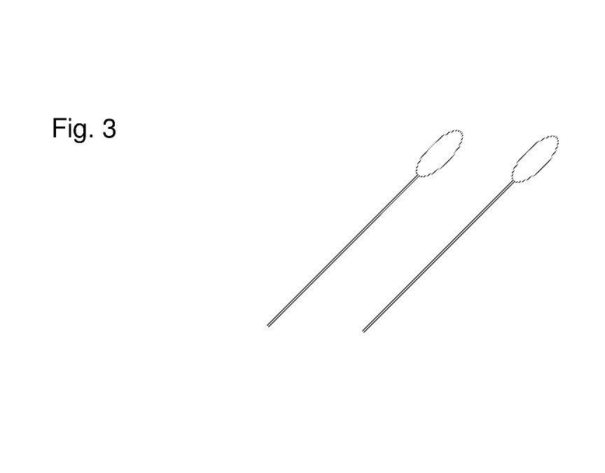

In independent applicator embodiment, the two formulations are provided on physically separate applications, e.g., a swab or gauze pad (FIG. 3). The independent applicators and their formulations may also be covered with a protective film that can be removed prior to use.

A formulation may be placed directly on a non-absorbent portion of the body if appropriately formulated as a gel, ointment, or cream. Alternatively, formulations may be absorbed into an absorbent portion of the body, (e.g., if formulated as a liquid).

Formulations in kits may be disposed in any suitable container. Examples include cotton swabs, cotton balls, gauze pads, bandages, wooden sticks, vials, squeeze tubes, capsules, and syringes. Kits may also include a barrier that will adhere to the tissue being treated. Suitable barriers include gauze, cotton, and plastics. Such barriers may be adhered to skin or oral mucosa using known temporary adhesives. Absorbent materials, (e.g., gauze), may also naturally adhere to oral mucosa without use of an adhesive.

Various topical formulations and dosages of anesthetics are known in the art. The invention will employ a formulation suitable for the location of administration and a dosage sufficient to induce loss of tactile, temperature, or taste sensation in a sensitive subject. For example, using a reference model, lidocaine may be administered as an ointment at 1-10%, (e.g., 5%), and benzocaine may be administered as a gel at 10-30%, (e.g., 20%), or using a control model, lidocaine may be administered as an ointment at 1-10%, (e.g., 5%), and a control of the base for the lidocaine without the anesthetic.

When administered orally, the area of application may first be dried, (e.g., by blotting or spraying with air), prior to administering the anesthetics.

Probes for tactile, temperature, or taste sensitivity may also be provided with devices or kits of the invention. One probe may be used for each location, but separate probes are preferable. Alternatively, an integrated probe with two prongs spaced for each location may be employed. Appropriate materials for tactile sensitivity are known in the art, e.g., wood, metal, and plastic. For temperature sensitivity, the probe may be a thermally conductive material, e.g., metal, that is heated or cooled to the desired temperature. Typically, such probes are heated or cooled to the desired temperature, e.g., refrigerator, freezer, oven, or water bath. Alternatively, probes may be heated or cooled thermoelectrically, by chemical reaction, or by liquids circulating in the probe. Heated probes may also include a resistive heating element. The probes may also expel heated or cooled gas or liquid (e.g., air or water), or the probes may include an edible solid material that produces a warm or cold feeling upon dissolution in the mouth. The temperature of the probes is typically at least 10 degrees Fahrenheit warmer or colder than the body temperature of the subject. Probes for taste will typically be made of an inedible material, such as wood, paper, plastic, or metal. The probes will include a taste agent, e.g., in liquid, gel, or dissolvable form, that produces a sweet, sour, salty, or bitter taste.

Metrics of Effectiveness:

Any differences in sensitivity can then be determined by the subject. For example, the subject can describe any difference in feeling of cold, heat, taste, numbness, puffiness, or pins and needles between the two locations. Under some circumstances, the two locations may also be probed, (e.g., by a blunt probe, pin, or heated or cooled probe), to aid in determining difference in sensation in the two areas. The subject can also indicate the sensation using a numerical scale, such as on a visual analog scale as modified for the tactile, temperature, or taste being employed as the metric.

Determining the effectiveness occurs after administration of the two formulations, e.g., up to 2 minutes after.

In one embodiment, the metric is the difference in tactile sensation. When the second formulation includes a second anesthetic, a difference in the tactile sensation between the first and second locations indicates that the first anesthetic is ineffective in the subject. When the second formulation does not include an anesthetic, no difference in the tactile sensation between the first and second locations indicates that the first anesthetic is ineffective in the subject. In certain embodiments, the determining may include questioning the subject regarding feeling of numbness, puffiness, or pins and needles. Alternatively, the determining includes applying pressure or a pin prick to the first and second regions.

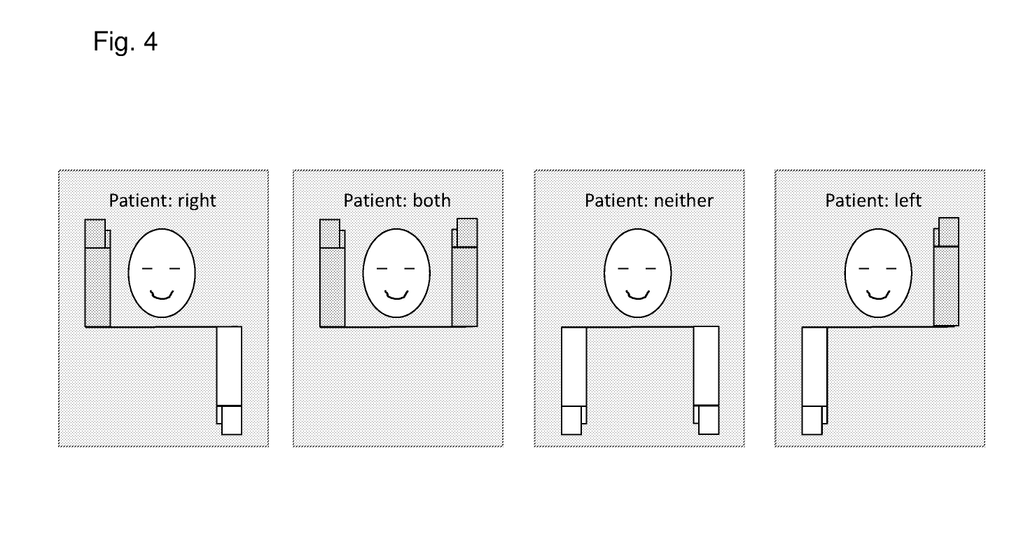

In another embodiment, the metric is temperature sensitivity. In this embodiment, probes that are warmer or colder than the body temperature are employed. For example, a kit may include 2 pieces of thermally conductive material (e.g., metal) suitable for use in humans. The thermally conductive material is preferably slow to adjust to room temperature. The probes may be cooled to ˜32 degrees Fahrenheit, and the probes may be applied to the locations in the mouth treated with the two formulations. The subject may then be asked to indicate by raising arms to indicate which side feels cold (FIG. 4): (1) Left colder; (2) Both sides cold (both arms raised); (3) Right colder; (4) Neither cold (both arms down). The kits are stable at room temperature, and only need to be refrigerated so that the metal or plastic pieces are cold. For temperature sensitivity and no anesthetic in the second formulation, if the perception of temperature is the SAME between the first and control regions that indicates that the first anesthetic is ineffective in the subject. In the case of the second formulation having a second anesthetic, if the perception of temperature is DIFFERENT between the first and reference regions that indicates the test anesthetic is ineffective in the subject.

In another embodiment, the metric is taste sensitivity. In this embodiment, taste agents are applied to the two locations, and the subject is asked whether there is a different in taste. For taste sensitivity and no anesthetic in the second formulation, if the taste is the SAME between the first and control regions that indicates that the first anesthetic is ineffective in the subject. In the case of the second formulation having a second anesthetic, if the taste is DIFFERENT between the first and reference regions that indicates the test anesthetic is ineffective in the subject.

Double-Blind Mechanism for Determining Effectiveness:

Directions on how the test subject is to indicate the side where they feel the effect (see FIG. 4A), together with a recording mechanism that avoids confusion about left and right (for example, allowing the invention to be used with younger children) and my left/your left confusion between subject and user may be provided. Furthermore, a peel off or scratch off “key” that indicates the interpretation for each of the 4 possible responses for this particular combination of materials and left-right orientations of the kit may also be provided, allowing the user to collect the responses in a manner that is double-blinded. In addition, the subject may be asked about the difference in sensation they are experiencing, reflecting that the ineffectiveness may not be absolute.

### Single Formulation Embodiments

The invention may also be employed with a single formulation including the first anesthetic. In these embodiments, the formulation may include a visual indicator, as described above. The formulation may be applied by any suitable device as described herein, e.g., swab or gauze pad or a single applicator as shown in FIG. 3. Determination of effectiveness may be performed using any of the metrics provided herein, tactile, temperature, or taste sensitivity. Kits including a single formulation may include any of the probes for tactile, temperature, or taste sensitivity as described herein. The contacting for determining tactile, temperature, or taste sensitivity can be performed only on the location treated with the formulation or on both the location of the formulation and an untreated location, as described herein when two formulations are employed. When only the location treated is tested, the subject can respond to contacting with a probe by indicating the absence of the feeling, perception of temperature change, or taste. When the treated location and an untreated location are tested, the sensitivity can be determined in the same manner as described herein for two formulations where the second formulation does not include anesthetic.

### Uses for the Invention

The determination that an anesthetic is ineffective for a subject is useful in several contexts. For example, a subject for whom a particular anesthetic is ineffective should be administered an alternative anesthetic prior to any medical, surgical, or dental procedure requiring anesthesia. Two other conditions, attention disorders and PMS, may also be treated as described below.

Attentional Disorders:

Attention deficit is a finding present in many disorders, and it is likely that abnormalities in many different genes can produce the finding of attention deficit. The standard treatment for attention deficit disorder using drugs that act on biogenic amine neurotransmission has led to a presumption that attention deficit disorder involves disturbances in biogenic amine neurotransmission. The existence of individuals with an attention disorder ascribable to a peripheral channelopathy adds a different mechanism of action to consider. An ability to treat an attention disorder in some individuals with different therapeutics, diet, or supplements could be a useful treatment option, particularly in light of recent concerns about side effects of drugs targeting biogenic amine neurotransmission.

Many families have been identified with attention deficit that are also insensitive to lidocaine (Segal et al. Journal of Child Neurology 22 (2007) 1408-1410; Segal Pediatric Neurology 51 (2014) 15-16). The families display features reminiscent of hypokalemic periodic paralysis, such as amelioration by potassium and exacerbation by sodium or glucose. The triggers for the sensory overload described by subjects correspond to those described in hypokalemic periodic paralysis (NaCl, and large carbohydrate meals) and other circumstances known to lower serum potassium (menstruation and diarrheal illnesses). As in hypokalemic periodic paralysis, potassium levels were not lower than those of unaffected subjects; instead, symptoms occurred during normal physiological fluctuations of serum potassium that would not cause symptoms in the average person. Because lidocaine injected peripherally acts on sodium channels in peripheral sensory pathways, this suggests an abnormality in this disorder expressed in a peripheral sensory pathway. Although attention deficit disorder is often presumed to have a central basis, overstimulation in a peripheral sensory pathway is another mechanism that could, in some forms of attention deficit disorder, explain the sensory overstimulation. Although this familial attention deficit with lidocaine ineffectiveness is found in less than half of people with attention deficit, it provides a manner to identify a subset of individuals suffering from attention deficit whose symptoms may be helped by specific therapeutics or dietary changes.

Accordingly, the invention provides methods for diagnosing subjects at risk for or diagnosed with a sub-type of attention disorder, (e.g., ADD or ADHD). Subjects that are identified as having lidocaine ineffectiveness may then be treated appropriately. In particular, the diet of such subjects may be modified to be low in sugar and sodium and/or administering potassium or drugs that modulate levels of potassium to the subject. Further treatments include aldosterone receptor antagonists, such as eplerenone or spironolactone, that increase potassium levels.

Premenstrual Syndrome:

As discussed above, women for whom lidocaine is not effective may not present with an attentional disorder but may instead have premenstrual syndrome. Accordingly, women suffering from premenstrual syndrome may be tested for the effectiveness of the anesthetic, (e.g., lidocaine). If ineffective, the premenstrual syndrome may be treated with potassium supplementation or drugs that modulate levels of potassium to the subject, such as a diuretic, e.g., a potassium sparing diuretic. Further treatments may include anti-inflammatory drugs, (e.g., an NSAID), or a stimulant, (e.g., caffeine).

1. A kit comprising:
i) an aliquot of a first formulation for topical administration comprising a first anesthetic; and
ii) an aliquot of a second formulation for topical administration wherein the second formulation comprises either a second anesthetic different from the first anesthetic or no anesthetic, and wherein the first formulation and second formulation are visually distinguishable when applied to a subject.

2. The kit of claim 1, wherein the second formulation does not comprise an anesthetic.

3. The kit of claim 1, wherein the second formulation comprises the second anesthetic.

4. The kit of claim 1, wherein the aliquot of the first and/or second formulation is disposed in gauze or a swab.

5. The kit of claim 1, further comprising a physical barrier configured to adhere topically to a subject.

6. The kit of claim 5, wherein the physical barrier comprises an absorbent material.

7. The kit of claim 1, wherein herein the first anesthetic is lidocaine.

8. The kit of claim 3, wherein the second anesthetic is benzocaine.

9. The kit of claim 1, furthering comprising a probe for tactile, taste, or temperature sensitivity.

10. The kit of claim 1, wherein the first formulation and second formulation are visually distinguishable by color, reflectivity, light scattering, opacity, or inclusion of particles.

11. A device comprising:
i) a body having first and second regions, where the first and second regions are physically separated;
ii) an aliquot of a first formulation for topical administration comprising a first anesthetic and disposed in the first region;
iii) an aliquot of a second formulation for topical administration and disposed in the second region, wherein the second formulation comprises either a second anesthetic different from the first anesthetic or no anesthetic.

12. The device of claim 11, wherein the body comprises a strip having a first face and the first and second regions are disposed on the first face.

13. The device of claim 12, wherein the strip further comprises at least one barrier located between the first and second regions.

14. The device of claim 11, wherein the body is configured for placement in the mouth of a subject.

15. The device of claim 14, wherein the body is configured for placement from the lip to the gum.

16. The device of claim 14, wherein the body is configured for placement from the upper lip to the upper gum.

17. The device of claim 14, wherein the body is shaped to accommodate a buccal frenulum.

18. The device of claim 11, wherein the body is configured for placement on the tongue, wherein the first and second regions are on the left and right side of the tongue.

19. The device of claim 11, wherein the body further comprises a protective sheet covering the first and/or second region, wherein the protective sheet is removed prior to use.

20. The device of claim 11, wherein the first anesthetic is lidocaine.

21. The device of claim 11, wherein the second formulation does not comprise an anesthetic.

22. The device of claim 11, wherein the second formulation comprises the second anesthetic.

23. The device of claim 22, wherein the second anesthetic is benzocaine.

24. The device of claim 11, wherein the first formulation and second formulation are visually distinguishable when applied to a subject

25. The device of claim 11, wherein the first formulation and second formulation are visually distinguishable by color, reflectivity, light scattering, opacity, or inclusion of particles.

26. A method of determining the effectiveness to a first anesthetic, the method comprising the steps of:
i) applying an aliquot of a first formulation topically to a first location of a subject, wherein the first formulation comprises a first anesthetic;
ii) applying an aliquot of a second formulation topically to a second location of the subject, wherein the second location is physically separated from the first region and the second formulation comprises either a second anesthetic different from the first anesthetic or no anesthetic; and
iii) determining tactile, taste, or temperature sensation at the first and second locations, wherein, when the second formulation comprises the second anesthetic, a difference in the sensation between the first and second locations indicates that the first anesthetic is ineffective for the subject, and, when the second formulation comprises no anesthetic, no difference in the sensation between the first and second locations indicates that the first anesthetic is ineffective for the subject.

27. The method of claim 26, wherein the first anesthetic is lidocaine.

28. The method of claim 26, wherein the second formulation does not comprise an anesthetic.

29. The method of claim 28, wherein the second formulation comprises the second anesthetic.

30. The method of claim 29, wherein the second anesthetic is benzocaine.

31. The method of claim 26, wherein the first and second locations are in the mouth of the subject.

32. The method of claim 26, wherein the first and second locations are on the tongue.

33. The method of claim 26, wherein the first and second locations are from the lip to the gum.

34. The method of claim 26, wherein step (iii) comprises applying pressure, pin prick, or elevated or reduced temperature to the first and second locations.

35. The method of claim 26, wherein step (iii) comprises applying a temperature probe to the first and second locations.

36. The method of claim 35, wherein the temperature probe is at least 10 degrees Fahrenheit colder than subject body temperature.

37. The method of claim 26, wherein step (iii) comprises applying a taste agent to the first and second locations.

38. The method of claim 37, wherein the taste agent is sweet, sour, salty, or bitter.

39. The method of claim 26, wherein step (iii) comprises determining the sensation at the first and second locations up to two minutes after steps (i) and (ii).

40. The method of claim 26, wherein the first formulation and second formulation are visually distinguishable when applied to a subject.

41. The method of claim 40, wherein the first formulation and second formulation are visually distinguishable by color, reflectivity, light scattering, opacity, or inclusion of particles.

42. The method of claim 26, further comprising, if the first anesthetic is ineffective for the subject, administering another anesthetic to the subject prior to a medical, dental, or surgical procedure.

43. The method of claim 26, wherein the subject is diagnosed with an attentional disorder.

44. The method of claim 43, further comprising, if the first anesthetic is ineffective for the subject, administering an aldosterone receptor antagonist to the subject.

45. The method of claim 44, wherein the aldosterone receptor antagonist is eplerenone or spironolactone.

46. The method of claim 43, further comprising, if the first anesthetic is ineffective for the subject, administering a low sugar and/or low sodium diet to the subject.

47. The method of claim 43, further comprising, if the first anesthetic is ineffective for the subject, administering potassium to the subject.

48. The method of claim 26, wherein the subject is diagnosed with premenstrual syndrome.

49. The method of claim 48, further comprising, if the first anesthetic is ineffective for the subject, administering a diuretic to the subject.

50. A method of treating an attentional disorder comprising administering to a subject in need thereof a therapeutically effective amount of an aldosterone receptor antagonist, a low sugar and/or low sodium diet, and/or potassium, wherein a first anesthetic is ineffective for the subject.

51. A method of treating premenstrual syndrome comprising administering to a subject in need thereof a therapeutically effective amount of a diuretic or potassium, wherein a first anesthetic is ineffective for the subject.

52. The method of claim 50 or 51, wherein the first anesthetic is lidocaine.

53. A kit comprising an aliquot of a formulation for topical administration comprising an anesthetic and a visual indicator of application to a subject.

54. The kit of claim 53, wherein the indicator is of color, reflectivity, light scattering, or opacity, or comprises visually identifiable particles.

55. The kit of claim 53, wherein the anesthetic is lidocaine.

56. The kit of claim 53, further comprising a probe for tactile, taste, or temperature sensitivity.

57. A kit comprising an aliquot of a formulation for topical administration comprising an anesthetic and a probe for tactile, taste, or temperature sensitivity.

58. The kit of claim 57, wherein the anesthetic is lidocaine.

59. A method of determining the effectiveness to an anesthetic, the method comprising the steps of:
i) applying an aliquot of a formulation topically to a first location of a subject, wherein the formulation comprises an anesthetic; and
ii) determining tactile, taste, or temperature sensation at the first location relative to a second location not treated with the formulation, wherein no difference in the sensation between the first and second locations indicates that the first anesthetic is ineffective for the subject.

60. The method of claim 59, wherein the anesthetic is lidocaine.

61. The method of claim 59, wherein the first and second locations are in the mouth of the subject.

62. The method of claim 59, wherein the first and second locations are on the tongue.

63. The method of claim 59, wherein step (ii) comprises applying pressure, pin prick, or elevated or reduced temperature to the first and second locations.

64. The method of claim 59, wherein step (ii) comprises applying a temperature probe to the first and second locations.

65. The method of claim 64, wherein the temperature probe is at least 10 degrees Fahrenheit colder than subject body temperature.

66. The method of claim 59, wherein step (ii) comprises applying a taste agent to the first and second locations.

67. The method of claim 66, wherein the taste agent is sweet, sour, salty, or bitter.

68. The method of claim 59, wherein step (ii) comprises determining the sensation at the first and second locations up to two minutes after step (i).

69. A method of determining the effectiveness to an anesthetic, the method comprising the steps of:
i) applying an aliquot of a formulation topically to a location of a subject, wherein the formulation comprises an anesthetic; and
ii) determining tactile, taste, or temperature sensation at the location, wherein a reduction in the sensation at the location indicates that the first anesthetic is ineffective for the subject, wherein the subject is diagnosed with an attentional disorder or premenstrual syndrome.

70. The method of claim 69, wherein the anesthetic is lidocaine.

71. The method of claim 69, wherein the location is in the mouth of the subject.

72. The method of claim 69, wherein the location is on the tongue.

73. The method of claim 69, wherein step (ii) comprises applying pressure, pin prick, or elevated or reduced temperature to the location.

74. The method of claim 69, wherein step (ii) comprises applying a temperature probe to the location.

75. The method of claim 74, wherein the temperature probe is at least 10 degrees Fahrenheit colder than subject body temperature.

76. The method of claim 69, wherein step (ii) comprises applying a taste agent to the location.

77. The method of claim 76, wherein the taste agent is sweet, sour, salty, or bitter.

78. The method of claim 69, wherein step (ii) comprises determining the sensation at the location up to two minutes after step (i).
